(19) United States
(12) Plant Patent Application Publication
(10) Pub. No.: US 2002/0152509 P1
(43) Pub. Date: Oct. 17, 2002

(54) Miniature rose variety 'POULgo001'

(75) Inventors: L. Pernille Olesen, Fredensborg (DK); Mogens N. Olesen, Fredensborg (DK)

Correspondence Address: Poulsen Roser Pacific, Inc., 2550 Old Military Road, Central Point, OR, 97502, US

(73) Assignee: Poulsen Roser ApS.

(21) Appl. No.: 09/776,560

(22) Filed: Feb. 1, 2001

(51) Int. Cl. A01H 5/00

(52) U.S. Cl. PLT/117

(57) ABSTRACT

A new miniature rose which has abundant, white flowers and attractive foliage. This new and distinct variety has shown to be uniform and stable in the resulting generations from asexual propagation.

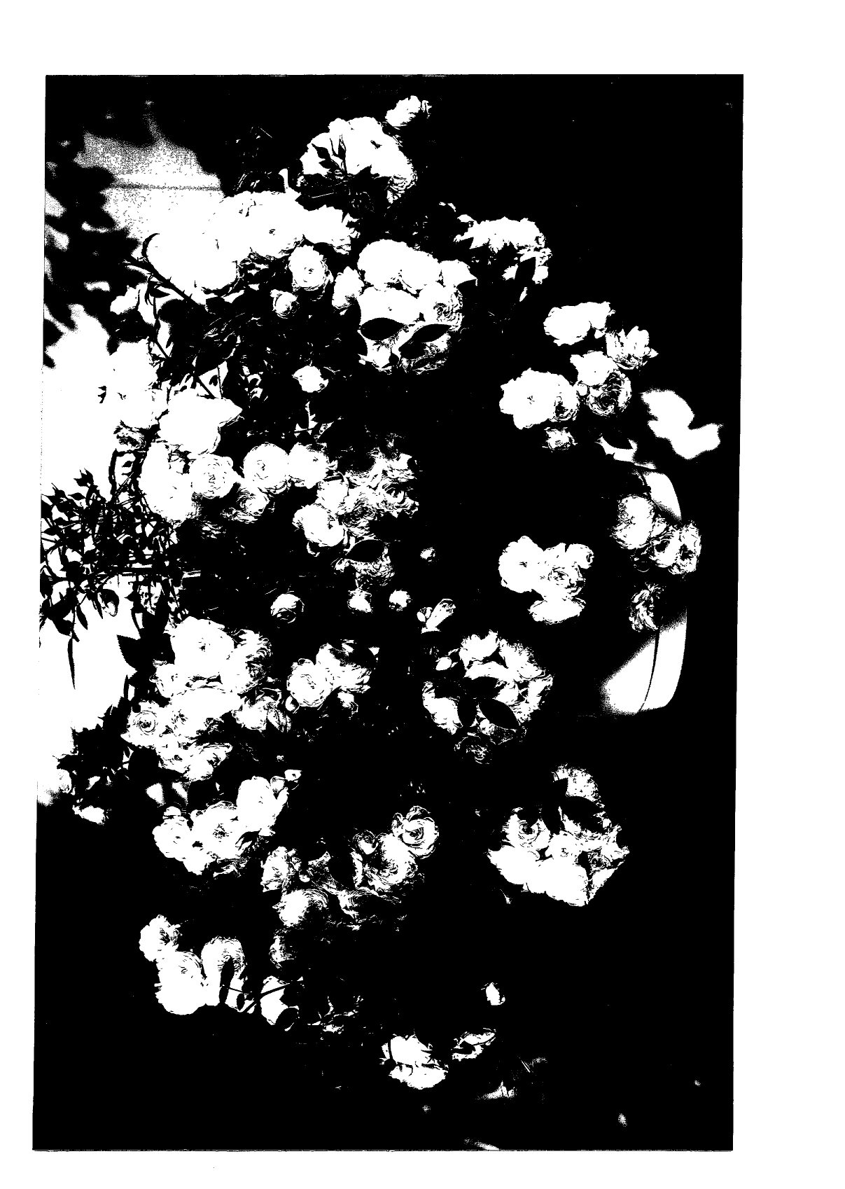

### SUMMARY OF THE INVENTION

The present invention constitutes a new and distinct variety of miniature rose plant which originated from a controlled crossing between an unnamed and unpatented seedling and ‘POULrijk’, described and illustrated in Plant patent application No. Ser. 09/261,445 dated Mar. 3, 1999. The two parents were crossed during the summer of 1994 and the resulting seeds were planted in a controlled environment in Fredensborg, Denmark. The new variety is named ‘POULgo001’.

The new rose may be distinguished from its seed parent, an unnamed seedling, by the following combination of characteristics:

1. The unnamed seed parent exhibits pink blooms, while ‘POULgo001’ has white blooms.

2. The unnamed seed parent is a miniature rose with an upright habit, whereas ‘POULgo001’ is a miniature rose with a pendulant habit.

The new variety may be distinguished from its pollen parent, ‘POULrijk’ by the following combination of characteristics:

1. The pollen parent has pink blooms, while those of ‘POULgo001’ are white.

2. The pollen parent has smaller flowers than ‘POULgo001’.

The objective of the hybridization of this rose variety was to create a new and distinct variety for garden use with unique qualities, such as:

1. Uniform and abundant white flowers;

2. Vigorous, pendulant growth when propagated with cuttings;

3. Disease resistance.

4. A miniature rose suitable for use in hanging basket containers in the nursery industry.

This combination of qualities is not present in previously available commercial cultivars of this type and distinguish ‘POULgo001’ from all other varieties of which we are aware.

As part of their rose development program, L. Pernille Olesen and Mogens N. Olesen germinated the seeds from the aforementioned hybridization during winter of 1994 and conducted evaluations on the resulting seedlings in a controlled environment in Fredensborg, Denmark.

‘POULgo001’ was selected in the spring of 1995 by the inventors as a single plant from the progeny of the aforementioned hybridization.

Asexual reproduction of ‘POULgo001’ by traditional budding and rooted cuttings was first done by L. Pernille and Mogens N. Olesen in their nursery in Fredensborg, Denmark in July 1995. This initial and other subsequent asexual propagations conducted in controlled environments have demonstrated that the characteristics of ‘POULgo001’ are true to type and are transmitted from one generation to the next.

### BRIEF DESCRIPTION OF THE DRAWING

The accompanying color illustration shows as true as is reasonably possible to obtain in color photographs of this type the variety rose ‘POULgo001’. Specifically illustrated in SHEET 1 is a plant of ‘POULgo005’ growing in a 20-cm hanging basket.

### DETAILED DESCRIPTION OF THE VARIETY

The following is a description of ‘POULgo001’, as observed in its growth during trials at the testing station Prüfstelle Rethmar under the testing authority Bundessortenamtin in Hannover, Germany and at the inventor's nursery in Fredensborg, Denmark. Color references are made using The Royal Horticultural Society (London, England) Colour Chart, 1995, except where common terms of color are used.

For a comparison, several physical characteristics of the rose variety ‘POULterp’, a rose variety from the same inventors described and illustrated in U.S. Plant patent application Ser. No. 09/273,165 dated Mar. 19, 1999 are compared to ‘POULgo001’ in Chart 1.

CHART 1

| | ‘POULgo001’ | ‘POULterp’ |
|---|---|---|
| Color of open | White Group 155C | White Group 155B-C |
| flower, outer | | |
| side, middle | | |
| zone. | | |
| Flower | 50-80 mm | 35 mm |
| diameter | | |
| Plant growth | 40 cm (h) × 60 cm (w) | 45-60 cm (h) × |
| | | 120-150 cm (w) |

Parents:

Seed parent.—Unnamed seedling.

Pollen parent.—‘POULrijk’

Classification:

Botanical.—Rosa hybrida.

Commercial.—Miniature.

### FLOWER AND FLOWER BUD

Blooming habit: Continuous.

Flower bud:

Bud form.—Broad-ovate.

Sepals.—Weak appendages.

Receptacle.—Size: Very small to small.

Peduncle.—Surface: Few to medium abundance of hairs and prickles.

Borne.—Many flowering shoots.

Flower bloom:

Fragrance.—Weak.

Size.—Average flower diameter is 50-80 mm when open.

Form.—Shape of flower when viewed from the side: Upper part: Flat. Lower part: Flattened convex. Viewed from above: Irregularly rounded.

Petalaqe.—Double. Average range: 25-35 petals under normal conditions.

Color:

Petals.—Inner Side: Middle zone: White Group 155C. Marginal zone: White Group 155C. Basal petal spot: No coloration observed. Outer side: Middle zone: White Group 155c. Marginal zone: White Group 155c. Basal petal spot: No coloration observed.

General tonality.—On open flower White Group 155C.

Petals:

Petal reflex.—Weak.

Undulation of margin.—Weak.

Size.—Small.

Quantity.—Average.

Reproductive Organs:

Filaments.—Color: Green.

Hips.—Shape: Pitcher-shaped.

### PLANT

Plant growth.—Spreading. Procumbent to arching habit. Height 40-60 cm.

Stems:

Thorns.—Incidence: Few short prickles, many long prickles. Size: Small to medium. Shape: Concave.

Plant foliage:

Leaf size.—Small.

Glossy.—Weak glossiness.

Color.—Upper Leaf Surface: Dark green. Juvenile foliage: Exhibits anthocyanin on young shoots. Anthocyanin intonation: Color: Reddish brown.

Plant leaflets:

Leaflet.—Cross Section: Slight concave. Margin Undulation: Absent to very weak.

Terminal leaflet.—Length of blade: Short. Width of blade: Narrow. Shape: Rounded.

Disease resistance: Above average resistance to mildew, rust, black spot, and Botrytis under normal growing conditions in Hannover, Germany and Fredensborg, Denmark.

Cold hardiness.—

The variety ‘POULgo001’ has been found to be cold hardy in Fredensborg, Denmark; and Hannover, Germany.

1. A new and distinct variety of rose plant of the miniature rose class, substantially as herein illustrated and described as a distinct and novel rose variety due to its abundant white flowers, procumbent to arching habit, disease resistance, and extended period of bloom.
